(19) United States
(12) Patent Application Publication
(10) Pub. No.: US 2026/0208407 A1
(43) Pub. Date: Jul. 23, 2026

(54) INTELLIGENT BIOMATERIALS WHICH CREATE VARIOUS SHAPES THROUGH REMOTE FOAMING OF POLYMERS

(71) Applicant: UIF (University Industry Foundation), Yonsei University, Seoul (KR)

(72) Inventors: Sung Woon CHA, Seoul (KR); Donghwan LIM, Seoul (KR); Jin HONG, Pyeongtaek-si (KR); Kwanhoon KIM, Bucheon-si (KR)

(73) Assignee: UIF (University Industry Foundation), Yonsei University, Seoul (KR)

(21) Appl. No.: 19/422,783

(22) Filed: Dec. 17, 2025

(30) Foreign Application Priority Data
Jan. 22, 2025 (KR) 10-2025-0009776

(51) Int. Cl. B29C 44/12; B29C 44/34; B29K 33/00; B29K 67/00; B29K 101/12; B29K 105/04; B29K 105/20; B29K 705/02; B29K 705/08

(52) U.S. Cl. CPC B29C 44/1204; B29C 44/3415; B29K 2033/12; B29K 2067/04; B29K 2101/12; B29K 2105/04; B29K 2105/20; B29K 2705/02; B29K 2705/08

(57) ABSTRACT

A biocompatible intelligent material capable of creating various shapes through remote foaming of a polymer is disclosed. The material may comprise a gas-containing polymer; and a restoring member that is at least partially embedded within the polymer, is heatable by electromagnetic induction, and returns to a memorized shape upon heating.

### CROSS-REFERENCE TO RELATED APPLICATION

This application claims priority from Korean Patent Application No. 10-2025-0009776, filed on Jan. 22, 2025 in the Korean Intellectual Property Office, and all the benefits accruing therefrom under 35 U.S.C. 119. The contents of each of the above applications are incorporated herein in their entirety by reference.

### BACKGROUND

### Field

The present invention relates to a biocompatible intelligent material capable of creating various shapes through remote foaming of a polymer.

### Description of Related Art

The Microcellular Foaming Process (MCP) is a technology that dissolves inert gas within a polymer to form a cellular structure. In this process, the polymer absorbs the gas and then induces thermodynamic instability through heating and depressurization, allowing it to be transformed into a lightweight material with a fine cellular structure. MCP can contribute to reducing density while maintaining high strength, making it applicable in various industries. Particularly, when applied to the manufacturing of medical devices, it has the potential to provide lightweight characteristics while performing necessary functions within the body.

The heat generation technology using eddy currents is a method of generating heat within conductive materials through the phenomenon of electromagnetic induction. This technology is based on the principle that the eddy currents within the material generate a magnetic field, which in turn allows the material to be heated. When combined with conductive materials that consider biocompatibility, it is possible to locally control the temperature of medical devices implanted in the body.

A shape-memory alloy (SMA) refers to a material that can remember a deformed state at a specific temperature and can be restored to its original shape through heating. This alloy is primarily composed of a combination of nickel and titanium, and it possesses high strength and biocompatibility, making it highly applicable in the medical field. In particular, when applied to medical devices or drug delivery systems that require complex shapes, it can exhibit the property of deforming or restoring to the desired shape under specific conditions.

Conventional medical devices have had the issue of requiring mechanical devices or external manipulation when volume expansion or shape change is needed at a specific location. This has led to an increase in the size and complexity of medical devices, potentially causing discomfort during implantation within the body. Additionally, there are often difficulties in achieving precise position control or localized manipulation, which can frequently result in the medical functions not being performed efficiently.

### SUMMARY

The task that the present invention aims to solve is to provide a material that can simultaneously undergo foaming and shape restoration within the body. This can achieve both the lightweight design and functional integration of medical devices while enhancing usability and biocompatibility. Such technology can be particularly useful in fields where precise space creation and functional performance are required. The foaming process can expand physical space or protect tissue, and the shape restoration function can assist in improving the accuracy and effectiveness of medical devices.

In one aspect, the present invention provides a remote foaming material comprising a gas-containing polymer; and a restoring member that is at least partially embedded within the polymer, is heatable by electromagnetic induction, and returns to a memorized shape upon heating.

In one embodiment, the polymer may comprise polymethyl methacrylate (PMMA), polycaprolactone (PCL), or one or more thermoplastic polymers.

In one embodiment, the polymer may contain one or more of carbon dioxide or nitrogen dissolved therein and can foam upon heating.

In one embodiment, the restoring member may comprise a metal.

In one embodiment, the metal may comprise one or more of aluminum (Al) or a nickel-titanium alloy (Nitinol).

In one embodiment, when the remote foaming material is heated to a temperature sufficient to induce foaming while being positioned, bonded, or inserted at the target site, the polymer undergoes foaming simultaneously, and the restoring member can return to a memorized shape.

In another aspect, the present invention provides a method for manufacturing a remote foaming material, comprising: a foaming-polymer preparation step of preparing a gas-containing polymer; and a restoring-member embedding step of embedding a restoring member that is capable of being heated by electromagnetic induction and restores to a memorized shape upon heating, such that at least a portion of the restoring member is embedded within the polymer.

In one embodiment, the polymer may be prepared from one or more of polymethyl methacrylate (PMMA), polycaprolactone (PCL), or a thermoplastic polymer.

In one embodiment, the foaming-polymer preparation step may be prepared by dissolving one or more of carbon dioxide or nitrogen in the polymer.

In one embodiment, the restoring member may be prepared from metal.

In one embodiment, the metal may be prepared to include one or more of aluminum (Al) or a nickel-titanium alloy (Nitinol).

In one embodiment, the polymer may be prepared in two or more portions in the foaming-polymer preparation step.

In one embodiment, the restoring member can be formed between the polymer during the restoring-member embedding step.

In another aspect, the present invention provides a method for volume expansion and shape fixation using a remote foaming material, comprising: a first step of placing, bonding, or inserting the remote foaming material, which includes a gas-containing polymer; and a restoring member that is at least partially embedded within the polymer, is heatable by electromagnetic induction, and restores to a memorized shape upon heating; and a second step of heating the remote foaming material to a temperature sufficient to induce foaming while restoring the restoring member to the memorized shape through electromagnetic induction.

In one embodiment, the target site may be inside a living body.

In one embodiment, the living body may be a human.

In one embodiment, the living body may be an animal.

The present invention includes a technology that utilizes a composite made by combining aluminum and polymer. First, aluminum and polymer are combined, and then the composite is manufactured through a batch process utilizing carbon dioxide. This composite is implanted in the body, and after implantation, aluminum is heated and the polymer is foamed using electromagnetic induction technology. During the foaming process, the polymer expands in volume to effectively fill the necessary space, thereby enabling the performance of specific medical functions. This method can contribute to simultaneously securing space and providing functional support within the body.

The present invention also includes a technology that can perform medical functions through the combination of shape-memory alloys and polymers. First, the characteristics of the shape-memory alloy composed of nickel and titanium are designed to be physically blocked by the polymer, and the polymer treated using carbon dioxide is combined with the shape-memory alloy. Subsequently, the composite implanted in the body heats the shape-memory alloy through electromagnetic induction, causing the polymer to foam. In this process, the shape-memory alloy is restored to the desired shape, while the foamed polymer performs drug delivery functions and simultaneously expands in volume to secure the necessary space. Additionally, the property of the polymer enveloping the shape-memory alloy can help minimize the physical impact on human organs.

The effect of the present invention is that it can enhance the efficiency and precision of medical devices by utilizing a functional material capable of simultaneously performing foaming and shape restoration. Such materials can be accurately applied to the required locations within the body and enable space creation or tissue support through localized foaming. Furthermore, by utilizing the shape restoration properties, it increases the design flexibility of the devices and can meet various demands. For example, it provides the possibility of reducing the complexity of device manipulation during surgery or promoting stable operation within the body.

### BRIEF DESCRIPTION OF DRAWINGS

FIG. 1 is a diagram illustrating the foaming process that forms a cellular structure through the mixing of a polymer and an inert gas.

FIG. 2 is a diagram illustrating the foaming process on a laboratory scale.

FIG. 3 is a diagram illustrating the process of inducing foaming through electromagnetic induction using a conductive material and a polymer that has absorbed gas.

FIG. 4 is a diagram illustrating the process by which a composite combining Nitinol and polymer performs its function through foaming and shape recovery inside the body.

FIG. 5A is a diagram illustrating the process of generating heat necessary for foaming by inducing eddy currents within a conductive material.

FIG. 5B is a diagram illustrating the relationship between the heat required for foaming and physical variables.

### DETAILED DESCRIPTIONS

Hereinafter, the embodiments of the present invention will be described in detail with reference to the attached drawings. The present invention can undergo various modifications and can take on various forms; therefore, specific embodiments are illustrated in the drawings and described in detail in the text. However, this is not intended to limit the present invention to the specific disclosed forms, and it should be understood that all modifications, equivalents, or alternatives included within the spirit and technical scope of the present invention are encompassed. Similar reference numerals have been used for similar components while describing each drawing. In the attached drawings, the dimensions of the structures are shown enlarged for the sake of clarity of the present invention.

The terms used in this application are employed solely for the purpose of describing specific embodiments and are not intended to limit the present invention. Singular expressions include plural expressions unless the context clearly indicates otherwise. In this application, terms such as “comprising” or “having” are intended to specify the existence of features, numbers, steps, actions, components, or combinations thereof as described in the specification, and should not be understood as excluding the presence or potential addition of one or more other features, numbers, steps, actions, components, or combinations thereof. In the context of this specification, terms such as “about” may refer to approximately ±1%, about ±2%, about ±3%, about ±4%, about ±5%, about ±6%, about ±7%, about ±8%, about ±9%, or about ±10% of the values specified in the specification.

Additionally, the description of one aspect of the present invention may be similarly applied to the same or similar configurations or terms in the description of other aspects.

Unless otherwise defined, all terms used herein, including technical or scientific terms, shall have the same meaning as understood by a person of ordinary skill in the relevant technical field. Terms that are defined in commonly used dictionaries should be interpreted to have meanings consistent with their context in the relevant technology, and unless explicitly defined in this application, they shall not be interpreted in an idealized or overly formal sense.

The remote foaming material according to an embodiment of the present invention may comprise a gas-containing polymer; and a restoring member that is at least partially embedded within the polymer, is heatable by electromagnetic induction, and returns to a memorized shape upon heating.

In the context of this specification, the term “foaming” of polymers refers to the process of inducing thermodynamic instability, which allows the gas dissolved within the polymer to expand and form a cellular structure. This foaming can help increase the volume of the polymer, thereby filling the desired space or providing physical support.

As long as it is capable of foaming, the material of the polymer is not specifically limited. In one embodiment, the polymer may comprise polymethyl methacrylate (PMMA), polycaprolactone (PCL), or one or more thermoplastic polymers.

The advantages of polymethyl methacrylate (PMMA) are that it possesses both high transparency and strength, making it potentially applicable in various fields. Additionally, PMMA offers excellent processability and stability, which makes it useful in applications requiring precise designs, such as medical devices. In particular, it has the characteristic of forming a uniform cellular structure when absorbing gas and foaming, which may make it suitable for remote foaming materials.

Polycaprolactone (PCL) is a biodegradable polymer with a low melting point and biocompatibility, making it safe for use within the body. PCL gradually decomposes over an extended period and possesses characteristics that allow it to be utilized as a drug delivery medium. Additionally, it demonstrates stable performance during the foaming process after gas absorption, which may be advantageous for the fabrication of medical devices. In particular, it can provide properties suitable for tissue regeneration or implantable devices.

A thermoplastic polymer refers to a polymer material that can melt and be molded when heat is applied, and solidifies into its original shape upon cooling. The advantages of thermoplastic polymers include the ability to be processed into various forms and a high potential for recyclability. These characteristics provide flexibility during the foaming and shape restoration processes, which can contribute to expanding the diversity of medical device designs. Additionally, through physical stability and ease of processing, they may support precise design capabilities.

Other non-limiting examples of possible polymers include polyethylene (PE), polypropylene (PP), polyurethane (PU), polycarbonate (PC), polyamide (PA), polyoxymethylene (POM), polytetrafluoroethylene (PTFE), polyisobutylene (PIB), polysulfone (PSU), polyvinyl chloride (PVC), polyethylene terephthalate (PET), polybutadiene (PBD), polystyrene (PS), acrylonitrile butadiene styrene (ABS), ethylene vinyl acetate (EVA), polylactic acid (PLA), polyglycolic acid (PGA), polyethylene oxide (PEO), poly(dodecamethylene adipate) (PDDA), polyimide (PI), polyacrylonitrile (PAN), polyethylene naphthalate (PEN), polyoxybenzoate (POB), polymethylpentene (PMP), polybenzimidazole (PBI), polycarbonate ethylene oxide (PC-EO), poly(ethylene-co-acrylic acid), polysulfone amide (PSA), and polyketone (PK).

Meanwhile, the remote foaming material of the present invention may be used when implanted inside a living body. In this case, the polymer may need to be biocompatible. Non-limiting examples of biocompatible polymers include polycaprolactone (PCL), polylactide (PLA), polyglycolic acid (PGA), polylactide-co-glycolide (PLGA), polyethylene glycol (PEG), hydroxyethyl cellulose (HEC), carboxymethyl cellulose (CMC), chitosan, alginate, hyaluronic acid, zein, gelatin, collagen, fibrin, dextran, polyvinyl alcohol (PVA), polyethylene oxide (PEO), polycarbonate urethane (PCU), polyimide (PI), polyurethane (PU), polysulfone (PSU), polytetrafluoroethylene (PTFE), polyamide (PA), polypropylene (PP), polyethylene (PE), polysaccharide, starch, silk, and bioelastin.

In the present invention, the role of the polymer is to provide space creation and physical support through foaming and/or to maintain biocompatibility that allows for safe operation within the body. The polymer has the property of dissolving gas and expanding, which can meet functional requirements such as tissue protection. Additionally, there is a possibility that it may buffer external forces during the foaming process or act as a drug delivery mediator at specific locations.

The principle by which the polymer foams is not particularly limited. In one embodiment, the polymer can foam upon heating with one or more gases, such as carbon dioxide or nitrogen, dissolved in it. The principle of foaming in a gas-containing polymer is based on inducing thermodynamic instability, which causes the dissolved gas within the polymer to expand, resulting in the formation of a fine cellular structure. As the polymer matrix softens through heating, the gas can diffuse and grow within the polymer, leading to volume expansion. This foaming process can be adjusted according to the polymer's glass transition temperature (Tg), the solubility of the gas, and external conditions. This principle may be useful for securing physical space through foaming or for forming lightweight structures with desired properties.

Therefore, the type of gas is not specifically limited, and non-limiting examples of other possible gases include helium (He), argon (Ar), krypton (Kr), xenon (Xe), hydrogen (H₂), oxygen (O₂), methane (CH₄), ethylene (C₂H₄), propane (C₃H₈), butane (C₄H₁₀), acetylene (C₂H₂), nitrous oxide (N₂O), ammonia (NH₃), carbon disulfide (CS₂), fluorocarbons (e.g., CF₄, C₂F₆), chlorinated hydrocarbons (e.g., CCl₄, C₂Cl₆), carbon monoxide (CO), formaldehyde (HCHO), acrylonitrile (ACN), methylene chloride (CH₂Cl₂), sulfur dioxide (SO₂), hydrofluoric acid (HF), hydrochloric acid (HCl), nitrogen trifluoride (NF₃), hydrogen sulfide (HS₂), and dimethyl ether (DME). These gases may be selectively used depending on the properties of the polymer and the required foaming characteristics. However, when used in biological applications, the type of gas may be somewhat limited.

In the context of this specification, the term “shape memory” refers to the property of a material that allows it to return to its original shape at a specific temperature. Shape memory is primarily activated by heat and can assist in restoring the deformed structure to recover or maintain the functionality of the device. This property can be useful in designing devices with complex shapes or utilizing variable structures according to medical requirements. This enables stable and effective operation even after implantation within the body.

In the present invention, the role of the restoring member is to implement the physical form and function required at a specific location through the foaming of the polymer and shape restoration. The restoring member maintains the memorized shape and can contribute to enhancing the precision and reliability of the device by restoring it through heating. Additionally, the restored shape can perform additional functions such as tissue protection, space creation, or drug delivery, making it suitable for various medical needs.

In one embodiment, the restoring member may include a metal. The advantage of the metal is that it has high thermal and electrical conductivity, enabling efficient heating through electromagnetic induction. Additionally, metals exhibit excellent strength and durability, providing structural stability, making them suitable for use as a restoring member. These characteristics can help precisely control the shape restoration process of the remotely foamed material and enhance reliability.

In one embodiment, the metal may comprise one or more of aluminum (Al) or a nickel-titanium alloy (Nitinol). The advantages of aluminum include its lightweight nature and high thermal conductivity, as well as its excellent heating efficiency through electromagnetic induction. Aluminum has excellent corrosion resistance and can be reliably used for implantation in the body due to its biocompatibility. Additionally, its good machinability makes it suitable for various composite designs, and it may serve as a cost-effective option.

The advantage of Nitinol is that it provides both shape-memory effect and superelastic properties simultaneously. This material can restore its deformed state to its original shape at a specific temperature, making it suitable for the design of complex medical devices. It has excellent biocompatibility and durability, allowing for safe use in implantable devices within the body. Additionally, it offers the benefit of precise heating and restoration through thermal and electromagnetic induction.

Non-limiting examples of other possible metals include iron (Fe), copper (Cu), gold (Au), silver (Ag), platinum (Pt), palladium (Pd), titanium (Ti), magnesium (Mg), chromium (Cr), zinc (Zn), nickel (Ni), cobalt (Co), molybdenum (Mo), tungsten (W), iridium (Ir), ruthenium (Ru), osmium (Os), rhodium (Rh), gallium (Ga), indium (In), tin (Sn), bismuth (Bi), zirconium (Zr), hafnium (Hf), vanadium (V), tantalum (Ta), cerium (Ce), lanthanum (La), neodymium (Nd), or alloys of two or more of these. These metals each possess unique physical and chemical properties and may be selectively utilized as restoring members in foamed materials.

The polymer expands space and provides physical support through foaming, while the restoring member can restore the memorized shape to maintain a precise structure. This embedded structure opens up the possibility of achieving multifunctional performance that is difficult to provide with a single material. Additionally, the manner in which the polymer envelops the restoring member can help minimize tissue damage during implantation.

In the context of this specification, heating by electromagnetic induction refers to the process of generating eddy currents to raise the temperature of a conductive material. This process is carried out through an external electromagnetic field, allowing for precise control of the device's temperature. Since electromagnetic induction operates in a non-contact manner, it enables safe and efficient heat generation from a distance. For example, it allows for safe and efficient heat generation within the body. This can simultaneously activate the foaming of the polymer and the shape restoration of the restoring member.

The reason the remote foaming material of the present invention can foam remotely is that it generates heat in a non-contact manner by utilizing electromagnetic induction technology. This allows for control from outside the device and enables localized operation only at the desired location.

The way the remote foaming material of the present invention operates is not specifically limited. In one embodiment, when the remote foaming material is heated to a temperature sufficient to induce foaming while being placed, bonded, or inserted at a target site, the polymer foams, and simultaneously, the restoring member can return to a memorized shape. By operating in this manner, the remote foaming material can perform necessary functions precisely and efficiently at specific locations. The foaming process can secure physical space or support tissue through the volume expansion of the polymer, and the shape restoration of the restoring member can maintain the structural stability of the device and enable additional functions. Furthermore, non-contact heating through electromagnetic induction provides the possibility of operating in areas within the body or regions that are difficult for a person to reach.

The polymer of the remote foaming material of the present invention does not exclude the addition of other substances. For example, when the remote foaming material is used for medical purposes and is implanted in a living body, substances that generate beneficial effects in the body through release from the polymer may be additionally included. Non-limiting examples of such substances include drugs, such as anti-inflammatory agents (e.g., dexamethasone), antibiotics (e.g., vancomycin), anticancer agents (e.g., doxorubicin), growth factors (e.g., VEGF, EGF), proteins (e.g., insulin), peptides (e.g., BMP-2), anticoagulants (e.g., heparin), analgesics (e.g., morphine), immunomodulators (e.g., cyclosporine), antioxidants (e.g., glutathione), antiviral agents (e.g., acyclovir), antifungal agents (e.g., fluconazole), lipid-lowering agents (e.g., statins), vitamins (e.g., vitamin D), minerals (e.g., calcium), biocompatible dyes (e.g., methylene blue), drug delivery carriers (e.g., liposomes), nanoparticles (e.g., silica nanoparticles), biodegradable microcapsules, biocompatible fillers, antibody-based therapeutics (e.g., monoclonal antibodies), nucleic acid-based therapeutics (e.g., siRNA, mRNA), prebiotics, probiotics, hyaluronic acid, alginate, chitosan, dextran, and collagen. These substances, when added to the polymer, provide the potential to support various medical purposes such as drug delivery, tissue regeneration, and infection prevention upon implantation in the body. Each substance may be selectively included based on its intended use and biocompatibility, and can exert beneficial effects depending on the in-body environment.

The advantages of the remote foaming material of the present invention described above are that it can enhance functional integration and efficiency in various engineering and medical applications. The characteristic of simultaneous foaming and shape restoration simplifies the design of complex engineering materials or medical devices and can reduce unnecessary volume or structure when inserted into hard-to-reach locations or within the body. Additionally, when used for medical purposes, the non-contact operation can minimize patient discomfort and contribute to increased precision in manipulation. These advantages suggest a potential for useful applications, particularly in surgeries requiring tissue reconstruction, drug delivery, or space creation.

On the other hand, the method for manufacturing a remote foaming material according to an embodiment of the present invention may include a foaming-polymer preparation step of preparing a gas-containing polymer; and a restoring-member embedding step of embedding a restoring member that is heatable by electromagnetic induction and restores to a memorized shape upon heating, such that at least a portion of the restoring member is embedded within the polymer.

The role of the foaming-polymer preparation step is to appropriately select a gas-containing polymer and prepare it in a state suitable for foaming. In this step, a gas can be dissolved considering the physical and chemical properties of the polymer. This allows the polymer to form a uniform cell structure during foaming and may enable it to operate safely within the body.

The role of the restoring-member embedding step is to combine the polymer and the restoring member to simultaneously achieve foaming and shape restoration functions. In this step, the restoring member can be appropriately positioned within or on the surface of the polymer, allowing it to be designed to return to its original shape when heated through electromagnetic induction. The restoring member operates stably even during the foaming process and can contribute to enhancing the structural integrity and functional diversity of the device. This step can play a crucial role in improving the integration and reliability of composite materials.

In one embodiment, the polymer may be prepared from one or more of polymethyl methacrylate (PMMA), polycaprolactone (PCL), or a thermoplastic polymer. In one embodiment, the foaming-polymer preparation step may be prepared by dissolving one or more of carbon dioxide or nitrogen in the polymer. In one embodiment, the restoring member may be prepared from a metal. In one embodiment, the metal may be prepared to include one or more of aluminum (Al) or a nickel-titanium alloy (Nitinol).

In one embodiment, the polymer may be prepared in two or more portions during the foaming-polymer preparation step. In one embodiment, the restoring member can be formed between the polymers during the restoring-member embedding step. The advantage of this method is that it allows for the independent optimization of the foaming and shape restoration functions. This approach can contribute to enhancing the physical properties required for foaming and the compatibility with the restoring member by separately designing the characteristics of each polymer. Additionally, by positioning the restoring member between the polymers, it provides the possibility of strengthening the structural stability and functionality of the composite.

Alternatively, in the restoring-member embedding step, the restoring member can be embedded within the polymer using an external force. This method has the advantage of allowing the manufacturing process to be carried out simply and quickly. Embedding using an external force enables the restoring member to be bonded to the polymer without the need for complex mechanical equipment, providing the potential to reduce manufacturing costs and time.

In one embodiment, in the restoring-member embedding step, a substance that has adhesive properties at least at some of the interfaces where the polymer and the restoring member are in contact can be formed. This can further enhance the durability and reliability of the composite. The adhesive layer can prevent physical separation or damage that may occur at the interface and improve the bonding strength between the restoring member and the polymer. This helps ensure that the restoring member operates stably during the foaming process and provides the possibility of maintaining functionality for an extended period after implantation in the body.

The advantages of the manufacturing method according to the embodiment of the present invention described above are that it can integratively implement the foaming and shape restoration functions while providing flexibility and efficiency in the manufacturing process. The stepwise process of appropriately combining the polymer and the restoring member optimizes the characteristics of each component and increases the possibility of precisely realizing the desired functions. Furthermore, the ability to custom design the structure of the composite allows for various applications according to engineering and medical requirements.

Meanwhile, the method for volume expansion and shape fixation according to an embodiment of the present invention may include: a first step of placing, bonding, or inserting at a target site a remote foaming material that comprises a gas-containing polymer and a restoring member that is at least partially embedded within the polymer, is heatable by electromagnetic induction, and returns to a memorized shape upon heating; and a second step of heating the remote foaming material to a temperature predetermined to induce foaming of the polymer while restoring the restoring member to the memorized shape through electromagnetic induction.

The role of the first step is to accurately place or bond the remote foaming material at the desired location, thereby creating initial conditions that allow for effective foaming and restoration processes. This step provides the possibility of preventing unexpected movement or loss of function in the operating environment by ensuring that the material is stably positioned at the target point. Additionally, it allows for manipulation and design in a suitable form according to the requirements of the target location, enabling precise application tailored to specific spaces.

The role of the second step is to activate the remote foaming material through electromagnetic induction, thereby simultaneously executing the volume expansion of the polymer and the shape restoration of the restoring member. This step operates in a non-contact manner, allowing for precise control while maintaining safety at the operating site. Through foaming, it is possible to secure the necessary space at the target site or support tissue, and the restoration of the restoring member can recover structural stability and functionality.

Examples of using the material of the present invention in a living body include surgical procedures, fixation of fracture sites, support for tissue defects, promotion of tissue regeneration, drug delivery media, implantable stents, soft tissue support, dura mater repair, vascular reconstruction, fixation of transplanted organs, drug delivery for cancer treatment, fluid collection, dental implant fixation, bioglue, microvascular connections, bladder repair, space filling after scar tissue removal, cartilage replacement, skin grafting, and compensation for organ damage. These applications present the potential to address various issues in medical environments where biocompatibility and stability are required.

Examples of using the material of the present invention in mechanical components include fixing pipes, mechanical vibration damping, enhancing thermal insulation structures, preventing leaks, repairing cracks, joining mechanical parts, preventing surface wear, restoring corroded metal components, sealing electronic equipment, securing cables, reducing vibrations in engine parts, maintaining balance in rotating devices, connecting parts of industrial robots, preventing air leakage, reinforcing cracks in building structures, sealing hydraulic systems, mechanical sealing, noise damping, and reinforcing energy absorption structures. These applications may contribute to enhancing mechanical reliability and maintenance efficiency in industrial environments.

In one embodiment, the subject may be a human. When used in humans, non-limiting examples of possible treatments or procedures include fixation of fracture sites, spinal disc reconstruction, joint replacement, support for tissue defect areas, drug delivery systems, protection of brain injury sites, fixation of cardiovascular stents, support for skin grafts, scar tissue restoration, filling spaces after tumor removal, promotion of soft tissue regeneration, dural repair, recovery from liver damage, organ fixation, assistance in abdominal hernia surgery, support for dental implants, assistance in varicose vein treatment, aid in cartilage regeneration, assistance in trauma treatment, and recovery from urinary system injuries. These applications may help meet various medical needs of humans.

In one embodiment, the living body may be an animal. When used in animals, non-limiting examples of possible treatments or procedures include fixation of fractured bones, assistance in the treatment of arthritis, recovery of tissue damage due to trauma, fixation of stents for animals, repair of skin damage, reinforcement of tissue after tumor removal, joint replacement, promotion of cartilage regeneration, support of internal organs, drug delivery systems, treatment of ocular trauma, assistance in abdominal hernia surgery, dental reinforcement devices, insertion of vascular stents, treatment of spinal injuries, assistance in bone grafting, support for wound suturing, abdominal tissue reinforcement, recovery from liver damage, and assistance in cardiovascular treatments. These applications suggest the potential usefulness in supporting rehabilitation and recovery in veterinary medicine.

The following describes embodiments of the present invention. However, the embodiments described below are merely some forms of the present invention, and the scope of the present invention is not limited to the embodiments provided herein.

### Microcellular Foaming Process (MCP)

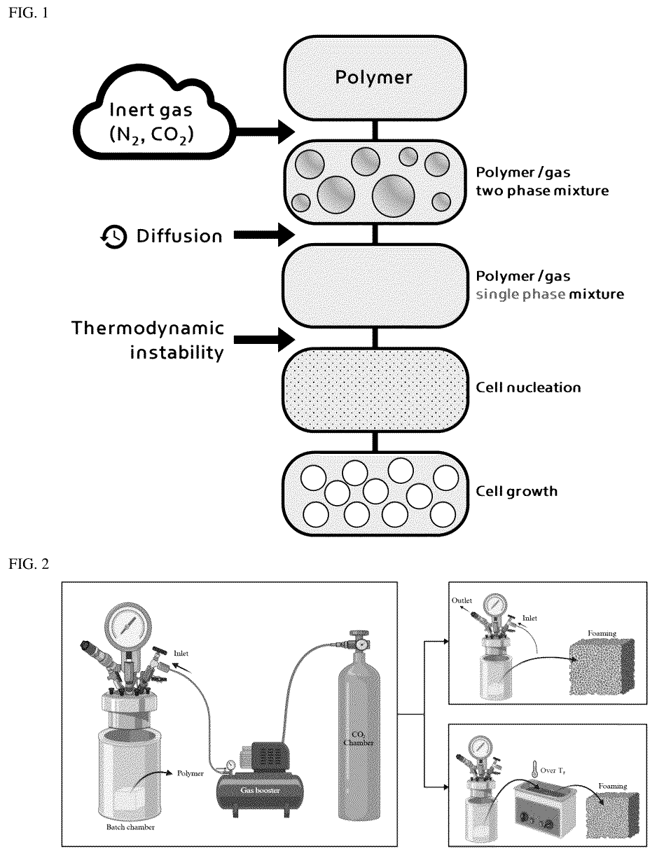

FIG. 1 is a diagram illustrating the foaming process that forms a cellular structure through the mixing of a polymer and an inert gas. This process consists of gas injection, diffusion, induction of thermodynamic instability, and the formation and growth of cell nuclei. Referring to FIG. 1, an inert gas such as nitrogen (N₂) or carbon dioxide (CO₂) is first injected into the free volume of the polymer, forming a two-phase mixture of polymer and gas. Subsequently, the gas diffuses into the polymer, transforming it into a uniform single-phase mixture. Once thermodynamic instability is induced, the formation of cell nuclei begins, and these nuclei grow, ultimately resulting in a foamed polymer containing cells with sizes of less than 100 micrometers. As a result, a structure is formed that contains over one billion cells per cubic centimeter. Through FIG. 1, the process of generating a uniform and fine cellular structure by utilizing the diffusion of the inert gas and thermodynamic instability can be confirmed.

Solid-state batch foaming process

FIG. 2 is a diagram illustrating the foaming process on a laboratory scale. This diagram includes the steps of absorbing an inert gas into a solid-state polymer and inducing foaming through heating and depressurization processes. Referring to FIG. 2, the polymer is first placed in a batch chamber, and an inert gas such as carbon dioxide (CO₂) or nitrogen (N₂) is injected at high pressure to allow the polymer to absorb the gas. Subsequently, the heating process induces the thermodynamic instability of the polymer, and finally, depressurization leads to the formation of bubbles within the polymer, resulting in foaming. This process can be summarized as creating a cellular structure and expanding the volume of the polymer to form a foamed polymer. Through FIG. 2, the laboratory process that integrates gas absorption, heating, and depressurization to induce foaming can be confirmed.

### Applications of MCP

The industrial applications of MCP can be utilized in various industries, including automotive parts, electronic products, and foam for footwear. It is particularly gaining attention for its mass production technology using Trexel's MuCell equipment. MCP has the advantage of achieving both low density and high strength simultaneously, which can enhance production efficiency through mass production capability and reduced cycle times. However, there are challenges in achieving a uniform cell structure, and issues such as surface roughness improvement and equipment cost burdens may act as limitations. This technology demonstrates potential for application in various industrial fields, including automotive interior materials, consumer electronics, and shoe cushioning.

### Implementation of Active Bending Using Shape-Memory Alloys and Polymer Composites

### Volume Expansion Using Dual-Material Composites

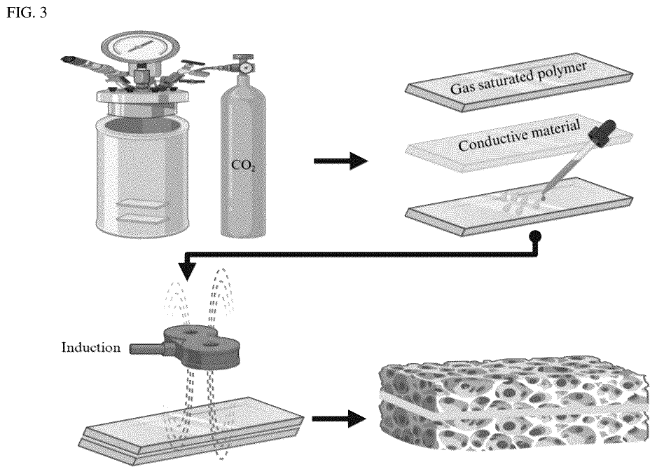

FIG. 3 is a diagram illustrating the process of inducing foaming through electromagnetic induction using a conductive material and a polymer that has absorbed gas. This diagram shows the steps from gas absorption, bonding with the conductive material, electromagnetic induction, to volume expansion through foaming. Referring to FIG. 3, the polymer is first prepared in a state containing a gas such as carbon dioxide (CO₂) and is then bonded with the conductive material. Subsequently, an electromagnetic induction device is used to generate eddy currents in the conductive material, producing heat. This heat causes the gas-absorbing polymer to foam locally, expanding in volume to form a structure that fills a specific space. The foaming is performed in a non-contact manner through electromagnetic induction and occurs locally only at the required locations. Through FIG. 3, the process of implementing foaming in a non-contact manner by utilizing the combination of the conductive material and the gas-absorbing polymer can be confirmed. This method allows for the securing of necessary space through volume expansion and offers the advantages of localized foaming and non-contact operation.

### Shape-Memory Alloy Nitinol

The shape-memory alloy Nitinol is an alloy composed of nickel and titanium in a 60:40 ratio, characterized by its ability to return to its original shape when heat is applied after plastic deformation. This alloy can memorize a new shape at high temperatures ranging from 450 to 600° C. and exhibits excellent biocompatibility, making it suitable for various medical applications. The shape change using electromagnetic induction heating is achieved by maintaining the deformed state at a low temperature (below 10° C.) and then applying heat using induction technology to restore the shape. In this process, the heating temperature is kept below 40° C. to ensure safety within the body. These characteristics can be utilized in biomaterials such as medical stents, and through the deformation and restoration processes, it can be applied in various forms and functions.

### Shape Transformation Using Nitinol and Polymer Composites

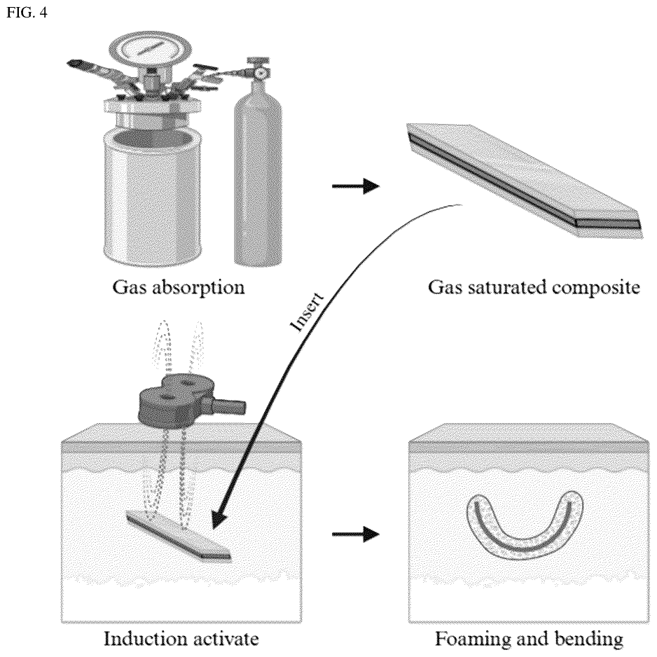

FIG. 4 is a diagram illustrating the process by which a composite combining Nitinol and polymer performs its function through foaming and shape recovery in vivo. This diagram shows the steps including the design of the composite, gas absorption, induction heating, and foaming and shape change. Referring to FIG. 4, Nitinol is first treated to remember a specific shape, then deformed into a flat shape and combined with PMMA. The combined composite is prepared in a gas-saturated composite state by absorbing carbon dioxide (CO₂) gas. Subsequently, the composite is implanted in the body, and the shape recovery of Nitinol is induced through induction heating using electromagnetic induction. In this process, the polymer undergoes foaming, resulting in a decrease in Young's modulus, and acquires soft and flexible properties, thereby protecting surrounding tissues and filling space. Through FIG. 4, the shape memory and recovery functions using the composite of Nitinol and polymer, as well as the tissue protection and space-filling functions through the foaming of the polymer, can be confirmed.

### Simulation of Temperature Prediction Model through Electromagnetic Induction

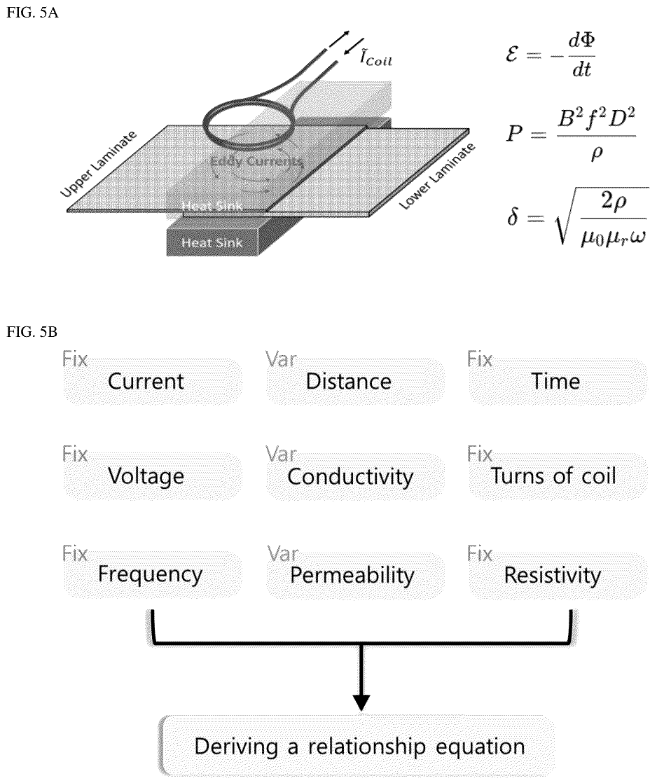

FIG. 5A is a diagram illustrating the process of generating heat necessary for foaming by inducing eddy currents within a conductive material. Referring to FIG. 5A, the magnetic field formed around the coil through which current flows induces eddy currents in the conductive material. These eddy currents generate heat within the conductive material, and the generated heat provides an appropriate temperature for polymer foaming. The equations presented in the diagram describe a method for calculating heat generation based on parameters, predicting the amount of heat and skin depth based on physical properties such as conductivity, frequency, permeability, and resistivity. This allows for quantitative control of heat generation in the conductive material.

FIG. 5B is a diagram illustrating the relationship between the heat required for foaming and physical variables. It shows the process of deriving the heat generation relationship equation through a combination of fixed and variable parameters. Referring to FIG. 5B, the fixed parameters include current, voltage, frequency, number of coil turns, time, and resistivity, while the variable parameters include distance, conductivity, and permeability. These parameters are used to quantitatively calculate the relationship between eddy currents and heat generation.

Through FIG. 5A and FIG. 5B, the principle of generating heat within a conductive material and the possibility of utilizing this for foaming control can be confirmed. By efficiently generating the heat required during the foaming process through eddy currents and enabling precise temperature control, the design supports achieving both in-body safety and optimal foaming performance simultaneously.

Although the above has been described with reference to a preferred embodiment of the present invention, those skilled in the art will understand that various modifications and changes can be made to the present invention without departing from the spirit and scope of the invention as set forth in the following claims.

What is claimed is:

1. A remote foaming material comprising:
a gas-containing polymer that is configured to foam upon heating; and
a restoring member at least partially embedded within the polymer, the restoring member comprising a material that is heatable by electromagnetic induction and that has a shape-memory property such that it returns to a memorized shape when heated.

2. The remote foaming material of claim 1,
wherein the polymer comprises polymethyl methacrylate (PMMA), polycaprolactone (PCL), or a thermoplastic polymer.

3. The remote foaming material of claim 2,
wherein the polymer contains dissolved carbon dioxide or nitrogen.

4. The remote foaming material of claim 1,
wherein the restoring member comprises a metal.

5. The remote foaming material of claim 4,
wherein the metal comprises aluminum (Al) or a nickel-titanium alloy (Nitinol).

6. The remote foaming material of claim 1,
wherein heating the remote foaming material to a temperature sufficient to induce foaming of the polymer also causes the restoring member to return to its memorized shape.

7. A method for manufacturing a remote foaming material, comprising:
a foaming-polymer preparation step of preparing a gas-containing polymer configured to foam upon heating; and
a restoring-member embedding step of embedding a restoring member, which comprises a material heatable by electromagnetic induction and having a shape-memory property, such that at least a portion of the restoring member is embedded within the polymer.

8. The method for manufacturing a remote foaming material of claim 7,
wherein the polymer is prepared from polymethyl methacrylate (PMMA), polycaprolactone (PCL), or a thermoplastic polymer.

9. The method for manufacturing a remote foaming material of claim 7,
wherein the foaming-polymer preparation step comprises dissolving carbon dioxide or nitrogen in the polymer.

10. The method for manufacturing a remote foaming material of claim 7,
wherein the restoring member is prepared from a metal.

11. The method for manufacturing a remote foaming material of claim 10,
wherein the metal comprises aluminum (Al) or a nickel-titanium alloy (Nitinol).

12. The method for manufacturing a remote foaming material of claim 7,
wherein, in the foaming-polymer preparation step, the polymer is prepared in two or more portions, and
wherein, in the restoring-member embedding step, the restoring member is positioned between the portions of the polymer.

13. A method for volume expansion and shape fixation using a remote foaming material, comprising:
a first step of placing, bonding, or inserting a remote foaming material comprising a gas-containing polymer configured to foam upon heating and a restoring member at least partially embedded within the polymer and comprising a material heatable by electromagnetic induction and having a shape-memory property, at a target site; and
a second step of heating the remote foaming material through electromagnetic induction to a temperature sufficient to induce foaming of the polymer and to cause the restoring member to return to its memorized shape.

14. The method of claim 13,
wherein the target site is inside a living body.

15. The method of claim 14,
wherein the living body is a human or an animal.
